(19) United States
(12) Patent Application Publication
(10) Pub. No.: US 2008/0213438 A1
(43) Pub. Date: Sep. 4, 2008

(54) Reduction of Benzene in Beverages and Compositions Therefor

(75) Inventors: Barry Edward Carter Williams, Dixon, IL (US); Lisa Ann Hayes, Ashton, IL (US); John Francis Tramontana, Naples, FL (US)

Correspondence Address: WELSH & KATZ, LTD, 120 S RIVERSIDE PLAZA, 22ND FLOOR, CHICAGO, IL, 60606, US

(73) Assignee: F.B.C. Industries, Inc.

(21) Appl. No.: 12/038,695

(22) Filed: Feb. 27, 2008

Related U.S. Application Data
(60) Provisional application No. 60/904,722, filed on Mar. 2, 2007.
(60) Provisional application No. 60/918,818, filed on Mar. 19, 2007.

(51) Int. Cl. A23L 2/42; G01N 33/14

(52) U.S. Cl. 426/231; 426/330.3

(57) ABSTRACT

Compositions useful for the reduction of the formation of benzene in beverages containing ascorbic acid in the presence of certain transition metals utilize a blend of aqueous solutions of sodium benzoate and potassium sorbate, in an approximate 50%/50% by weight solution. A method for reducing the formation of benzene in such beverages includes adding the blend to the beverage. Methods for reducing the degradation of sorbate solutions during storage using the compositions and for accelerating the formation of benzene are provided.

This application claims the benefit of U.S. Provisional Applications Nos. 60/904,722, filed Mar. 2, 2007, and 60/918,818, filed Mar. 19, 2007, both of which are incorporated herein by reference.

### BACKGROUND OF THE INVENTION

1. Field of the Invention

The present invention relates to the reduction of the formation of benzene in beverages, to compositions therefor, and to reduction in the degradation of sorbates; and more particularly to the reduction of the formation of benzene in beverages containing ascorbic acid and in the presence of a transition metal having an atomic number of from 21 to 30 in the form selected from the group consisting of the metal and alloys containing the metal, to compositions therefor, and to reduction of the degradation of sorbate solutions during storage.

2. Description of Related Art

It is common in the beverage industry to combine sodium benzoate and ascorbic acid (vitamin C) or sodium benzoate and juice containing natural ascorbic acid. If a small amount of a transition metal having an atomic number of from 21 to 30 in the form selected from the group consisting of the metal and alloys containing the metal, for example either copper or iron, is introduced to the beverage through a number of ways, to include water or other components, a chemical reaction can take place within the beverage container producing benzene, a known carcinogen. Benzene production in the presence of ascorbic acid was reported in a communication entitled “Benzene Production from Decarboxylation of Benzoic Acid in the Presence of Ascorbic Acid and a Transition-Metal Catalyst” in the Journal of Agricultural and Food Chemistry, Vol. 41, No. 5 (May 1993).

In addition, it has been reported, for example in an article by John N. Sotos, in Sorbate Food Preservatives, 1989, that aqueous solutions of sorbates, and particularly potassium sorbates, unlike the dry sorbates, are unstable and can rapidly degrade. The present invention seeks to reduce the formation of benzene in beverages containing ascorbic acid and reduce the degradation and polymerization of aqueous sorbate solutions during storage.

Thus, it would be advantageous to provide compositions which can be added to beverages, particularly beverages such as citrus juices, and the like, containing ascorbic acid, which reduce the formation of benzene, especially where the beverages will come into the presence of certain transition metals or their alloys. It would be further advantageous to extend the shelf-life of sorbate liquid solutions by providing an additive solution which can be added to the sorbate solution to reduce its degradation, for example through reduction of the formation of peroxides and reduction of polymerization of the sorbate in the solution.

### SUMMARY OF THE INVENTION

Embodiments of this invention provide compositions which when added to beverages containing ascorbic acid reduce the formation of benzene when the beverages come into contact with certain transition metals, such as copper and/or iron.

In another aspect of this invention compositions are provided which extend the shelf-life of liquid sorbate solutions by reducing the formation of peroxides and reducing polymerization of sorbate solutions, particularly aqueous solutions of potassium sorbate.

In particular, we have determined an aqueous solution of sodium benzoate and potassium sorbate is effective in reducing the formation of benzene. Unexpectedly, a blend of aqueous solutions of sodium benzoate and potassium sorbate, in an approximate 50%/50% by weight solution has been found to dramatically reduce the amount of benzene produced in a solution containing sodium benzoate and ascorbic acid.

It has also been unexpectedly discovered that the addition of an aqueous solution of sodium benzoate to an aqueous solution of potassium sorbate reduces the formation of peroxides of the sorbate and dramatically reduces polymerization of the sorbate solution. Preferably, an amount of an aqueous solution of sodium benzoate is added to an aqueous solution of a sorbate, particularly an aqueous solution of potassium sorbate, of approximately 50%/50% by weight of the benzoate solution to the sorbate solution.

The unexpected effectiveness of aqueous solutions of sodium benzoate and potassium sorbate as compared to sodium benzoate alone, for example, in a 1:1 ratio by weight of sodium benzoate and potassium sorbate, is demonstrated by a laboratory protocol that accelerates shelf life conditions and maximizes the amount of benzene produced in beverages-like matrixes which contain both sodium benzoate and ascorbic acid.

### BRIEF DESCRIPTION OF THE DRAWING

FIG. 1 is a graph illustrating various concentrations of benzene as a function of time.

### DETAILED DESCRIPTION OF THE INVENTION

It is preferable in accordance with this invention to utilize an aqueous blend of an aqueous solution of potassium sorbate and an aqueous solution of sodium benzoate that are of a grade generally recognized as safe (GRAS) for use in beverages for human consumption.

One embodiment of the invention is to utilize aqueous solutions of sodium benzoate and potassium sorbate at levels which, when blended, have a taste and odor acceptable in the beverage in which the blend is to be utilized.

Another embodiment is to utilize aqueous solutions of each of sodium benzoate and potassium sorbate which are blended, for example by mixing to achieve a homogenous product. In this embodiment, it is preferable to blend by mixing aqueous solutions of sodium benzoate and potassium sorbate which are FCC (Food Chemicals Codex) grade liquids and which are GRAS. Most preferably, in accordance with this invention, is the use of a blend of FCC grade aqueous sodium benzoate containing approximately 33% solids by weight and FCC grade aqueous potassium sorbate containing approximately 50% solids by weight, available from FBC Industries, Inc., Schaumburg, Ill. U.S.A. The two aqueous solutions are desirably present in ratios of from about 1:0 to about 1:5 sodium benzoate to potassium sorbate based on the weight of the solids of each ingredient and in aqueous solution of from about 40% to about 45% total solids by weight. Most preferably the FCC grade liquid aqueous solutions of sodium benzoate and potassium sorbate are present in a ratio of from about 1:0 to about 1.3 based on solids by weight. Particular weight FCC grade liquid solutions ratios of sodium benzoate to potassium sorbate obtained from FBC Industries, Inc. as noted above are 1:1 in a 40% solids aqueous solution, 1:1.15 in a 41.5% solid aqueous and 1:1.3 in a 44% solids aqueous solution.

### EXAMPLE 1

In the following example, premixed solutions were prepared as follows:

Buffer: 1.1500 grams of a solution of monosodium phosphate and water was prepared by mixing phosphoric acid with water to a pH of 2.996.

KS/SB Blend: a blended aqueous solution was prepared, by mixing 19.88 grams of aqueous FCC grade potassium sorbate containing approximately 50% solids by weight, available from FBC Industries, Inc., Schaumburg, Ill. USA, with 30.1225 grams of aqueous FCC grade sodium benzoate aqueous solution containing approximately 33% solids by weight, available from FBC Industries, Inc. (supra). 2.25 grams of the blended aqueous solution was poured into a glass container and mixed with water to obtain 100 milliliters (ml) of the KS/SB Blend.
A. 24 ml of Buffer, and 4 ml each of KS/SB Blend, ascorbic acid, water and copper sulfate were mixed to obtain a solution of 450 mg/kg by weight of each of potassium sorbate and sodium benzoate.
B. 24 ml of Buffer and 4 ml each of: an aqueous solution of 30.227 grams of aqueous FCC grade sodium benzoate containing approximately 33% solids by weight available from FBC Industries, Inc. (supra) and 20.4742 grams water, 2.303 grams of blended aqueous solution was poured in a glass container and mixed with water to obtain 100 milliliters; ascorbic acid; water; and copper sulfate were mixed to obtain a solution of 450 mg/kg of sodium benzoate.
C. 24 ml Buffer (adjusted to pH 3.009) and 4 ml each of: an aqueous solution of 2.7211 grams of aqueous FCC grade sodium benzoate containing approximately 33% solids by weight, available from FBC Industries, Inc. (supra) mixed with sufficient water to obtain 100 ml of solution; ascorbic acid; water and copper sulfate were mixed to obtain a solution of 900 mg/kg of sodium benzoate.

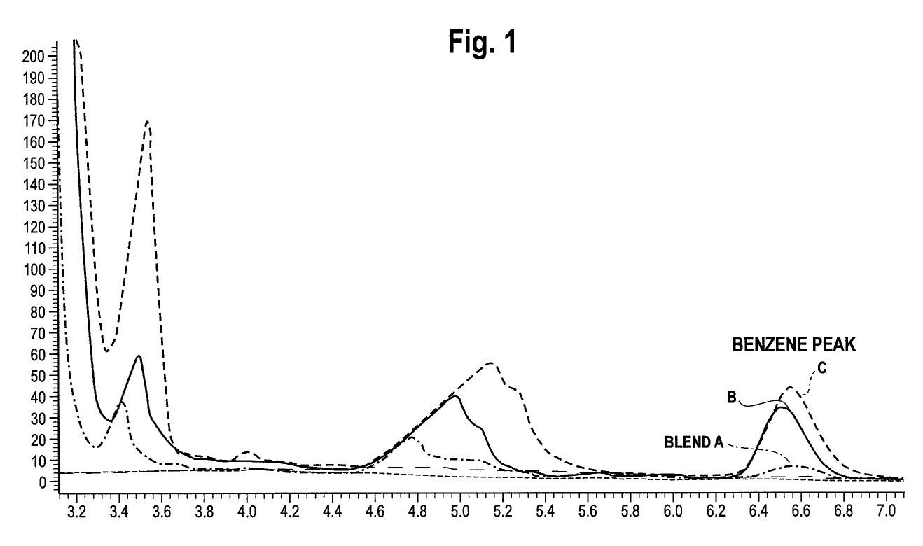

The solutions A, B and C were each heated in separate containers for three hours at a temperature of approximately 50° C. to accelerate the production of benzene. The solutions were submitted to high performance liquid chromatography with the production of benzene shown in the graph of FIG. 1, Benzene Production—Comparison KS/SB and SB:

C −900 mg/kg Sodium Benzoate
B −450 mg/kg Sodium Benzoate
A −450 mg/kg Sodium Benzoate and 450 mg/kg Potassium Sorbate

As shown in FIG. 1, the benzene level resulting from Formulation A was considerably less than that shown by Formulations B and C; B represents an equivalent level of sodium benzoate under identical conditions; C establishes that B is not anomalistic.

### EXAMPLE 2

Samples of aqueous solutions of potassium sorbate and of a blend of approximately 50% by weight of solids aqueous solution of potassium sorbate and approximately 50% by weight of solids aqueous solution of sodium benzoate were prepared by mixing, and retained in closed containers. After 6½ months the sample of the aqueous solution of potassium sorbate showed color degradation and increased viscosity, indicating polymerization had occurred; while after more than nine months the sample of the blend of the aqueous solutions of potassium sorbate and sodium benzoate showed no color change or increased viscosity, indicating no polymerization had occurred.

Example 2 demonstrates that the addition of an aqueous solution of sodium benzoate to aqueous solution of potassium sorbate, in approximately equal ratios by weight, dramatically reduced degradation and polymerization of the potassium sorbate solution, and therefor unexpectedly extended the shelf-life of the potassium sorbate solution.

These and other embodiments and advantages of the invention will be apparent to those skilled in the art from the above description and the appended claims.

What is claimed is:

1. A method for reduction of the formation of benzene in beverages in the presence of a transition metal having an atomic number of from 21 to 30 in the form selected from the group consisting of the metal and alloys containing the metal, comprising:
a. preparing an aqueous mixture of an aqueous solution of potassium sorbate and an aqueous solution of sodium benzoate; and
b. adding the mixture to a beverage which will contact the metal or an alloy containing the metal.

2. The method of claim 1, wherein the aqueous solutions of potassium sorbate and sodium benzoate are Food Chemicals Codex grade liquids and are of a grade generally recognized as safe for use in beverages for human consumption.

3. The method of claim 2, wherein the sodium benzoate and potassium sorbate are present at levels which when blended, has a taste and odor acceptable in the beverage in which the blend is utilized.

4. The method of claim 3, wherein the aqueous solution of sodium benzoate and the aqueous solution of potassium sorbate are blended.

5. The method of claim 4, wherein the aqueous solution of potassium sorbate and the aqueous solution of sodium benzoate are present in an approximately 1.0 to 1.5 ratio by weight of solids of the sodium benzoate to potassium sorbate and in an aqueous solution of from about 40% to about 45% total solids by weight.

6. The method of claim 4, wherein the aqueous solution of potassium sorbate and the aqueous solution of sodium benzoate are present in an approximately 1.0 to 1.3 ratio by weight of solids of the sodium benzoate to potassium sorbate and in an aqueous solution of from about 40% to about 45% total solids by weight.

7. A composition comprising a blend of an aqueous solution of potassium sorbate and an aqueous solution of sodium benzoate, in which the ratio of sodium benzoate to potassium sorbate is approximately 1.0 to 1.5 by weight of solids and in an aqueous solution of from about 40% to about 45% total solids by weight.

8. The composition of claim 7, wherein the blend of an aqueous solution of potassium sorbate and an aqueous solution of sodium benzoate is in a ratio of sodium benzoate to potassium sorbate of approximately 1.0 to 1.3 by weight of solids and in an aqueous solution of from about 40% to about 45% total solids by weight.

9. The composition of claim 7, wherein the aqueous solution of potassium sorbate and the aqueous solution of sodium benzoate is in a ratio of sodium benzoate to potassium sorbate of approximately 1 to 1 by weight of solids and in an aqueous solution of about 40% total solids by weight.

10. The composition of claim 7, wherein the aqueous solution of potassium sorbate and the aqueous solution of sodium benzoate is in a ratio of sodium benzoate to potassium sorbate of approximately 1.0 to 1.15 by weight of solids and in an aqueous solution of about 41.5% total solids by weight.

11. The composition of claim 7, wherein the aqueous solution of potassium sorbate and the aqueous solution of sodium benzoate is in a ratio of sodium benzoate to potassium sorbate of approximately 1.0 to 1.3 by weight of solids and in an aqueous solution of about 44% total solids by weight.

12. A method for extending the shelf-life of aqueous solutions of potassium sorbate comprising mixing an aqueous solution of sodium benzoate with the aqueous solution of potassium sorbate.

13. The method of claim 12, wherein the aqueous solution of potassium sorbate and the aqueous solution of sodium benzoate are present in a ratio of approximately 1.0 to 1.5 by weight of solids of the sodium benzoate to potassium sorbate and in an aqueous solution of from about 40% to about 45% total solids by weight.

14. The method of claim 12, wherein the aqueous solution of potassium sorbate and the aqueous solution of sodium benzoate is in a ratio of approximately 1.0 to 1.3 ratio by weight of solids of the sodium benzoate to potassium sorbate and in an aqueous solution of from about 40% to about 45% total solids by weight.

15. The method of claim 12, wherein the aqueous solution of potassium sorbate and the aqueous solution of sodium benzoate is in a ratio of sodium benzoate to potassium sorbate of approximately 1 to 1 by weight of solids and in an aqueous solution of about 40% total solids by weight.

16. The method of claim 12, wherein the aqueous solution of potassium sorbate and the aqueous solution of sodium benzoate is in a ratio of sodium benzoate to potassium sorbate of approximately 1.0 to 1.15 by weight of solids and in an aqueous solution of about 41.5% total solids by weight.

17. The method of claim 12, wherein the aqueous solution of potassium sorbate and the aqueous solution of sodium benzoate is in a ratio of sodium benzoate to potassium sorbate of approximately 1.0 to 1.3 by weight of solids and in an aqueous solution of about 44% total solids by weight.

18. A method of accelerating and maximizing the amount of benzene produced in beverages containing sodium benzoate and ascorbic acid, comprising mixing an aqueous solution of potassium sorbate with an aqueous solution of sodium benzoate, adding ascorbic acid and copper sulfate, heating the mixture to a temperature and for a period sufficient to accelerate the production of benzene, and determining the quantity of benzene produced in the mixture.

19. The method of claim 18, wherein the mixture includes a buffer of water and phosphoric acid mixed to a pH of approximately 3.

20. The method of claim 19, wherein the mixture of the aqueous solution of potassium sorbate and the aqueous solution of sodium benzoate comprises approximately 50% solids by weight of potassium sorbate and approximately 33% solids by weight of sodium benzoate.
